(19) United States
(12) Patent Application Publication
(10) Pub. No.: US 2022/0031570 A1
(43) Pub. Date: Feb. 3, 2022

(54) COMPONENT MIXING DEVICE AND COMPONENT MIXING METHOD

(71) Applicant: Alaxia SAS, Lyon (FR)

(72) Inventors: Julien Marine, Angers (FR); Michel Houdou, Angers (FR); Pascale Gaillard, Lyon (FR); Sandrine Perrotto, Fleurieu sur L'Arbresle (FR); Philippe Bordeau, St. Pierre de Chandieu (FR)

(21) Appl. No.: 17/295,613

(22) PCT Filed: Nov. 23, 2018
(86) PCT No.: PCT/FR2018/000258

§ 371 Date: May 20, 2021

(51) Int. Cl. A61J 1/20; A61J 1/16; B01F 15/00; B01F 15/02; B01F 15/04; A61K 38/40

(52) U.S. Cl. CPC A61J 1/2089; C12Y 111/01007; A61J 1/2037; A61J 1/2079; A61J 1/201; A61J 1/2058; B01F 15/00396; B01F 15/00357; B01F 15/0205; B01F 15/00974; B01F 15/0429; B01F 15/00344; A61K 38/40; C12Y 101/03004; A61J 1/165

(57) ABSTRACT

The present relates to a component mixing device comprising a central unit (10) and a disposable cartridge, said central unit (10) comprising a control unit, a plurality of vial positioning elements (13) and a cavity 14 for receiving said disposable cartridge, and said disposable cartridge (20) comprising a tubing circuitry adapted to put the components housed in said plurality of vials into communication for mixing, characterized in that the mixing device is adapted to put each vial into communication with each other through said tubing circuitry of said disposable cartridge (20) and mixing said components through an compressed air mechanism (12) located in said central unit (10) and controlled by said control unit.

### TECHNICAL FIELD

The present invention relates to a component mixing device and a component mixing method and more particularly to a component mixing device and a component mixing method capable of preparing an extemporaneous drug.

### BACKGROUND OF THE ART

Extemporaneous compounding is the preparation of a therapeutic product for an individual patient in response to an identified need. It is a practical way to have medicines supplied when there is no other option. For example, extemporaneous compounding may be useful for patients with dysphagia who are unable to swallow solid medications, when an appropriate dose or dosage form is not commercially available, when patients require an individualized dose, or when medicines must be delivered via nasogastric or gastrostomy tubes.

Active pharmaceutical ingredients can be incorporated into a wide array of products including creams, eye drops, nasal sprays, oral dosage forms or intravenous infusions. In general, products may be classified into simple or complex compounding. Simple compounding can be performed by any pharmacist and is a core competency of pharmacy training. Complex compounding requires additional training and evidence. In any case, compounding by the patient himself is usually highly discouraged.

Extemporaneously compounded/manufactured medicines may be useful when a required dose or dose form is unavailable commercially, or for individualized dosing. There are numerous established compounding formulae available, and new formulae may be developed with the help of formulation guidelines and professional advice. Also, short-term expiry dates are provided for compounded products unless their stability has been assessed.

On the other hand, extemporaneous preparation remains one of the highest risk preparative activities carried out in the pharmacy or by the patient himself. This is largely due to the dangers of using unlicensed medicines being amplified by the inherent risks associated with the pharmaceutical compounding process. This is evidenced by several reported errors connected to the use of extemporaneously prepared medicines, resulting in serious harm to patients.

It is also argued that this risk may be exacerbated by a declining expertise in pharmaceutics and formulation within the pharmacy profession. Compounding of oral medicines is often delegated to junior or trainee staff, and there is commonly no quality assurance system in place to support practice.

In addition, there appears to be a relationship between the environment in which medicines are prepared, the level of quality assurance applied to the processes involved and ultimately, the level of residual risk to patients. As one might expect extemporaneous preparation in the pharmacy or at home by the patient is considered to represent the lowest levels of quality assurance and the highest risk, whereas manufacture of licensed medicines provides the most robust assurance of quality, safety and efficacy. However, manufacture of promising medicines is not always possible, especially when using unstable active ingredients or reagents.

There is therefore a need for a device and a method providing a reliable and easy way to manufacture extemporaneous drugs at home by the patient, in the pharmacy or medical care centers and/or to give access to new active ingredients, not initially available, especially instable ones produced in situ.

The need for such extemporaneous drug preparation is particularly needed for preparing drugs which have a very short shelf life. For example, recently such a short shelf life medicine is in development for treating lung infections in Cystic fibrosis (CF) patients. Cystic fibrosis is a progressive, genetic disease that causes persistent lung infections and limits the ability to breathe over time.

In people with CF, a defective gene causes a thick, sticky buildup of mucus in the lungs, pancreas, and other organs. In the lungs, the mucus clogs the airways and traps bacteria leading to infections, extensive lung damage, and eventually, respiratory failure. In the pancreas, the mucus prevents the release of digestive enzymes that allow the body to break down food and absorb vital nutrients. People with cystic fibrosis are at greater risk of getting lung infections because thick, sticky mucus builds up in their lungs, allowing germs to thrive and multiply. Lung infections, caused mostly by bacteria, are a serious and chronic problem for many people living with the disease. Minimizing contact with germs is a top concern for people with CF.

The buildup of mucus in the pancreas can also stop the absorption of food and key nutrients, resulting in malnutrition and poor growth. In the liver, the thick mucus can block the bile duct, causing liver disease. In men, CF can affect their ability to have children.

Breakthrough treatments have added years to the lives of people with cystic fibrosis. Today the median predicted survival age is close to 40. This is a dramatic improvement from the 1950s, when a child with CF rarely lived long enough to attend elementary school. Because of tremendous advancements in research and care, new drug capable of treating CF have been developed.

In particular, a medicine made from an enzyme and a substrate but which is very unstable and needs to be prepared just before the inhalation was described WO 2013053777A1.

There is therefore a need for a component mixing device and a component mixing method which permits a patient to prepare his own extemporaneous antimicrobial drug.

Component mixing products and component mixing methods already exist. However, these kinds of devices use improper mechanisms such as pump and the like, which need to be lubricated and which present a risk of drug contamination.

In this regard, a primary object of the invention is to solve the above-mentioned problems and more particularly to provide component mixing product and component mixing method which can be easily and reliably used by a patient himself to prepare a proper extemporaneous drug.

### SUMMARY OF THE INVENTION

The above problems are solved by the present invention.

In a general manner, the present invention refers to a device permitting manufacturing and/or reconstituting a least one component of interest and preparing different predetermined quantities of the same, said component being purified or not, separately or in combination, in a simultaneous and repeated manner.

A first aspect of the invention is a disposable cartridge to be used with a separated central unit in a component mixing device, characterized in that it comprises a rigid microfluidic circuitry including at least three circuitry inlets each being adapted to enter in fluid communication with a vial to permit insertion of the content of each vial within said circuitry, at least one circuitry outlet for outputting a mixed product from the cartridge and a plurality of valves adapted to be controlled by a control unit of said central unit in order to create a specific path within said circuitry between said inlets and said outlet, wherein said circuitry is adapted to enter in fluid communication with a pressure gradient generating mechanism of said central unit in order to generate a circuitry content driving force within it.

According to a preferred embodiment of the present invention, the tubing circuitry is adapted to put at least a first and a second vials in fluid communication so as to mix the reagent content of one of them with the other of them. Advantageously, it further comprises at least one storing vessel for storing an intermediary product obtained through the mixing of the contents of at least two vials.

Preferably, it further comprises a microfilter for filtering at least one intermediary product.

Advantageously, the microfilter is located between the inlets and the storing vessel.

According to a preferred embodiment of the present invention, the filter is a dialysis microfilter adapted to switch between tangential and frontal filtration.

Preferably, the mixing process is carried out through the use of a pressure gradient which is generated by a compressed air mechanism located within said central unit which creates either overpressure or depression in at least one of the vials.

Advantageously, it further comprises at least one buffer tank connected to the vials or the filter.

According to a preferred embodiment of the present invention, the mixing process carried out within it is comprised in the group of a dilution, a reaction, a succession of reactions, an enzymatic cleavage, an elution and/or a purification,

A second aspect of the invention is a central unit to be used with the disposable cartridge of the first aspect in a component mixing device, characterized in that it comprises a plurality of vial positioning elements for receiving vials comprising specific reagents, a cavity for receiving said disposable cartridge, a control unit and a pressure gradient generating mechanism controlled by said control unit, wherein said pressure gradient generating mechanism is adapted to generate overpressure or depression in specific portions of said disposable cartridge to generate a reagent/product driving force.

Preferably, it comprises a movement mechanism adapted to move said vials into said cavity so as to enter in fluid communication with each other through said tubing circuitry of the disposable cartridge.

Advantageously, it further comprises in-air filters.

According to a preferred embodiment of the present invention, the control unit is adapted to control a desired quantity of each reagent according to a user's choice.

Preferably, the central unit comprises a temperature monitoring system to regulate temperature in said cavity.

In a preferable embodiment, the central unit comprises a screen for displaying successive steps of the mixing process.

Advantageously, the screen is adapted to warn said user upon a wrong placement of a vial.

A third aspect of the invention is a component mixing device comprising the disposable cartridge of the first aspect and the central unit of the second aspect, characterized in that the component mixing device is adapted to put specific vials into communication with each other through said tubing circuitry of said disposable cartridge and mixing said reagents according to a specified reacting process through an pressure gradient generating mechanism located in said central unit and controlled by said control unit.

A fourth aspect of the invention is a component mixing method comprising using the component mixing device of the third aspect and carrying out the following steps: placing a first vial comprising an aqueous solution of glucose, NaSCN and a buffer, a second vial comprising glucose oxidase and lactoperoxidase and a third vial comprising a lactoferrin component in respective of vial positioning elements, placing the disposable cartridge into the cavity, controlling said valves and said pressure gradient generating mechanism to first create a communication path between said first vial and second vial to mix their content in order to carry out a 2-step enzymatic reaction through the use of compressed air to synthetize HOSCN and/or OSCN⁻, then pass said OSCN⁻ solution through a dialysis microfilter and storing the resulting OSCN⁻ solution in a storing vessel, create a communication path between said third vial and said storing vessel to mix said lactoferrin with said OSCN⁻ solution through the use of compressed air so as to prepare an extemporaneous medicine for treating CF lung infections, and storing said extemporaneous medicine for treating CF lung infections into a syringe.

Preferably, the plurality of vials comprises at least three vials.

Advantageously, the third vial comprises a NaCl water diluted lactoferrin solution.

According to a preferred embodiment of the present invention, the plurality of vials comprises at least four vials wherein the third one comprises solid lactoferrin and the fourth one comprises NaCl in water.

A fifth aspect of the invention is a component mixing kit comprising the component mixing device of the third aspect and a plurality of vials, each vials being adapted to comprise a specific quantity of reagent for compounding a specific drug.

Preferably, the component mixing kit comprises one vial containing an enzyme, one vial containing a substrate and one vial containing lactoferrin and being adapted to compound a CF lung infections treating drug.

A sixth aspect of the invention is a use of component mixing kit of the fifth aspect for manufacturing an extemporaneous pharmaceutical formulation intended for the treatment of mucous microbial infections wherein the pharmaceutical formulation is a fluidic antimicrobial solution.

Preferably, the use of component mixing kit is for manufacturing an extemporaneous pharmaceutical formulation intended for the treatment of the lung infections, comprising OSCN⁻ ion and/or lactoferrin wherein the pharmaceutical formulation is in an inhalable form.

Preferably, the use of component mixing kit is for manufacturing an extemporaneous pharmaceutical formulation intended for the treatment of acute and chronic phases of the lung infections in cystic fibrosis, comprising OSCN⁻ ion and/or lactoferrin wherein the pharmaceutical formulation is in an inhalable form.

### BRIEF DESCRIPTION OF THE DRAWINGS

Further particular advantages and features of the invention will become more apparent from the following non-limitative description of at least one embodiment of the invention which will refer to the accompanying drawings, wherein

FIG. 1 represents a component mixing kit according to the present invention

FIG. 2 represent a view of a disposable cartridge according to a preferred embodiment of the present invention

FIG. 3 schematically represents a functional diagram of a component mixing kit according to the present invention;

FIGS. 4A and 4B schematically represent a movement of vials within a component mixing device of the present invention;

FIGS. 5A to 5D schematically represent several steps of a component mixing method within the disposable cartridge of the present invention.

### DETAILED DESCRIPTION OF THE INVENTION

The present detailed description is intended to illustrate the invention in a non-limitative manner since any feature of an embodiment may be combined with any other feature of a different embodiment in an advantageous manner.

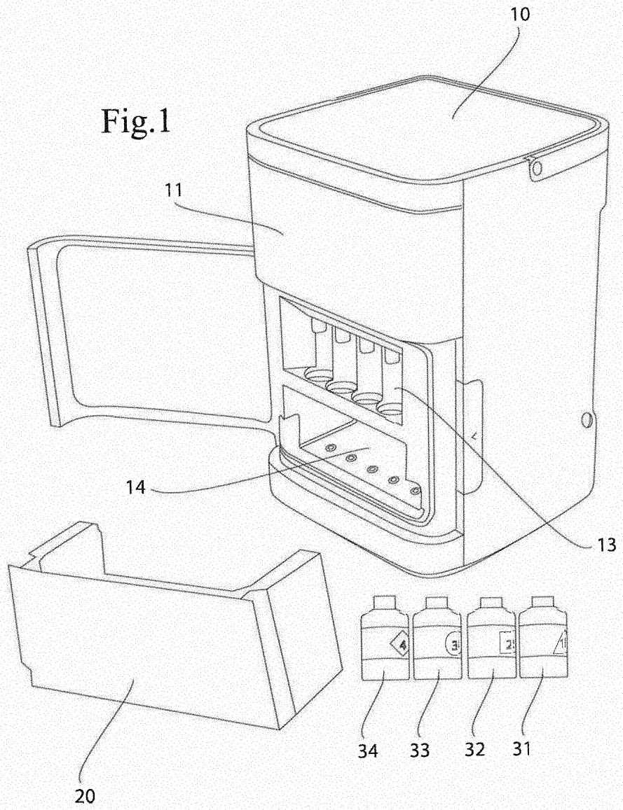

FIG. 1 shows an aspect of the invention which is a component mixing kit for preparing an extemporaneous drug and comprising a component mixing device 10, 20 of the present invention and a plurality of vials 31, 32, 33, 34, each vial being adapted to comprise a specific quantity of reagent for compounding a specific drug. As we will see later, the component mixing kit preferably comprises one vial 31 containing one or two enzymes and one vial 32 containing corresponding substrates, i.e. at least two vials 31, 32. Preferably but not necessarily it can comprise one or two more vials 33, 34 containing lactoferrin and possibly NaCl solution and being adapted to compound a CF lung infections treating drug.

The component mixing device comprises two main parts: a central unit 10 and a disposable cartridge 20 both being an object of the present invention. It is adapted to put the specific vials 31-34 into communication with each other through a tubing circuitry 21 of the disposable cartridge 20 and mixing reagents according to a specified reacting process through a pressure gradient generating mechanism 12 located in the central unit 10 and controlled by a control unit (not represented).

The central unit 10 according to a preferred embodiment of the invention generally comprises the whole control system, i.e. a CPU, a memory, the control unit, an embedded software, etc. adapted to control an operation of the present central unit 10. More particularly, it will control a desired quantity of each reagent to be sent within the tubing circuitry 21 for the compounding/mixing/preparation process and according to a specific timing according to a user's choice.

It also comprises a pressure gradient generating mechanism 12 which is adapted to generate overpressure or depression in specific portions of said disposable cartridge 20 to generate a reagent/product driving force such as a compressed air mechanism 12 which is used to drive the different fluids from vials 31-34 through the disposable cartridge 20 without the need of any pump inside the disposable cartridge 20, by creating either overpressure or depression in the desired location of the tubing circuitry 21 or within a specific vial 31-34.

It also comprises a plurality of vial positioning elements 13 adapted to receive the different vials 31-34 containing the reagents for manufacturing the selected drug. These vial positioning elements 13 can comprise some mismating or keying elements such that the vials 31-34 will not be placed in the wrong vial positioning element. These mismating or keying element may be of any type such as a color code, a shape fitting between the vial's bottleneck and the vial positioning element 13 a bar code reader within the vial positioning element and bar code on the vial, a chip/sensor system on the vial and its positioning element, etc. The form of the keying element is not crucial as long as it permits to warn a user that he placed the wrong vial in the wrong vial positioning element 13 or imped him to do so.

Correct placement of the respective vials 31-34 in the vial positioning element 13 is important since the control unit can operate the pressure gradient generating mechanism 12 as well as tubing circuitry valves 22 according to a specific placement of the vials 31-34.

The central unit 10 also comprises a display screen 11 which can be tactile. This screen 11 can show a user the process duration, any action required before/during/after the operation, a vial mispositioning warning, successive steps of the mixing process or the same. It also can present some control button to be pressed by the user to effect some action such as start, stop, eject or the same.

Finally, the central unit 10 comprises a cavity 14 which is adapted to receive a disposable cartridge 20. In this cavity 14, the central unit 10 presents some attachment mechanisms (not represented) that permit to fix a disposable cartridge 20 when inserted and also various air channels 15 located to mate some openings in the disposable cartridge 20 so as to create a fluid communication between the pressure gradient generating mechanism 12 and the tubing 21 of the disposable cartridge 20.

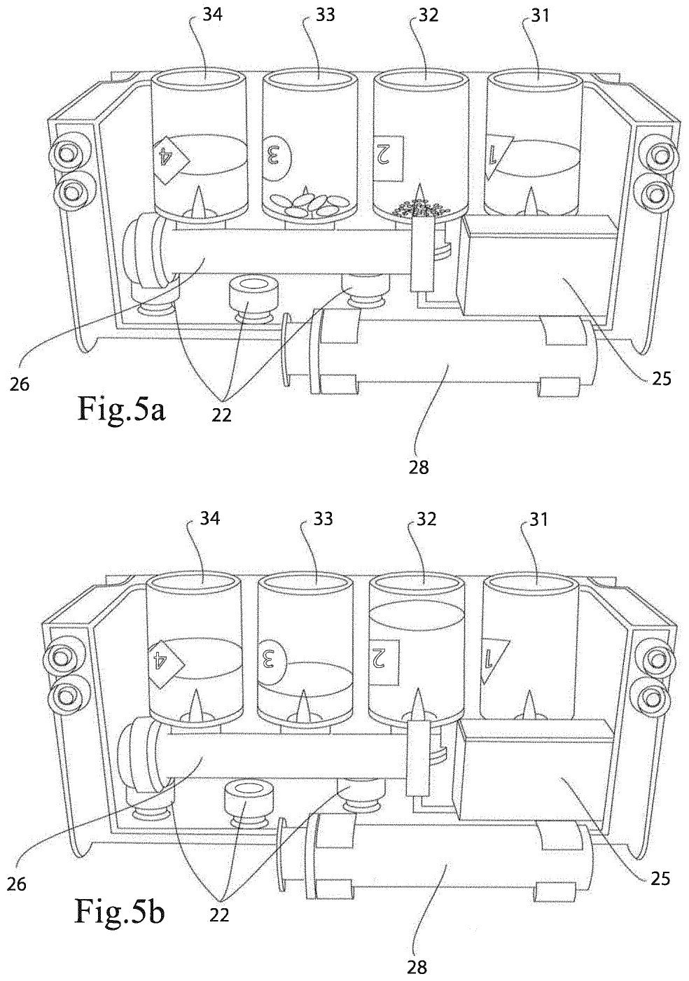

The central unit 10 also comprises a mechanism adapted to move the vials into said cavities so as to enter in fluid communication with each other through said tubing circuitry 21 of the disposable cartridge 20. FIGS. 4a and 4b show this movement, but it shall be understood that a movement in the other direction, i.e. from the bottom to the top could be possible in case of inverting the location of the vials 31-34 and the disposal element 20. Alternatively, it might be possible to have a mechanism that moves the disposable cartridge 20 instead of the vials 31-34 for achieving the same result. The main object here is that the central unit 10 comprises a moving mean which achieves a communication between the vials 31-34 and the tubing circuitry 21 of the disposable cartridge 20.

Although not represented, it is preferred that the central unit 10 comprises a temperature monitoring system to regulate temperature in said cavity 14, such as a cooling system for refrigerating said cavity 14 so that the different reagents and or active ingredients can have a slightly improved stability. Also, in order to obtain a sterilized atmosphere within mixing circuitry, which includes, the tubing circuitry 21 as well as the vials 31-34 and any portion of the disposable cartridge 20 in contact with the reagents, the intermediary products or the final product, it is preferred that each pressure gradient entry is provided with an in-air sterilizing filter 16. These filters may however be provided within the central unit air outlet and/or within the disposable cartridge air inlet. Providing them within the central unit air outlet is more cost-effective.

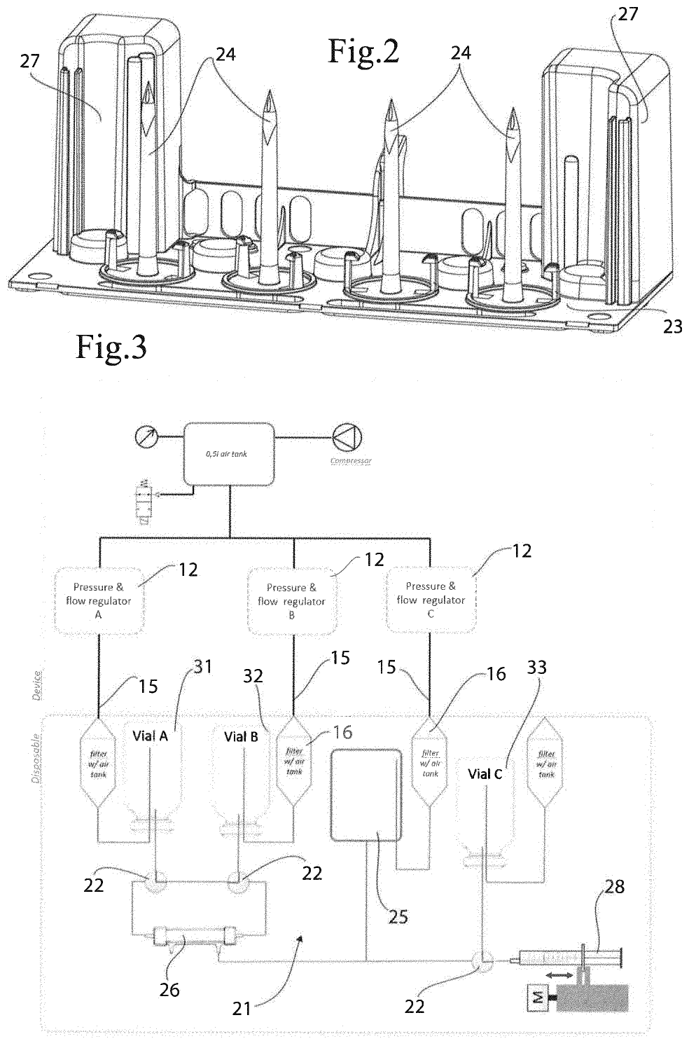

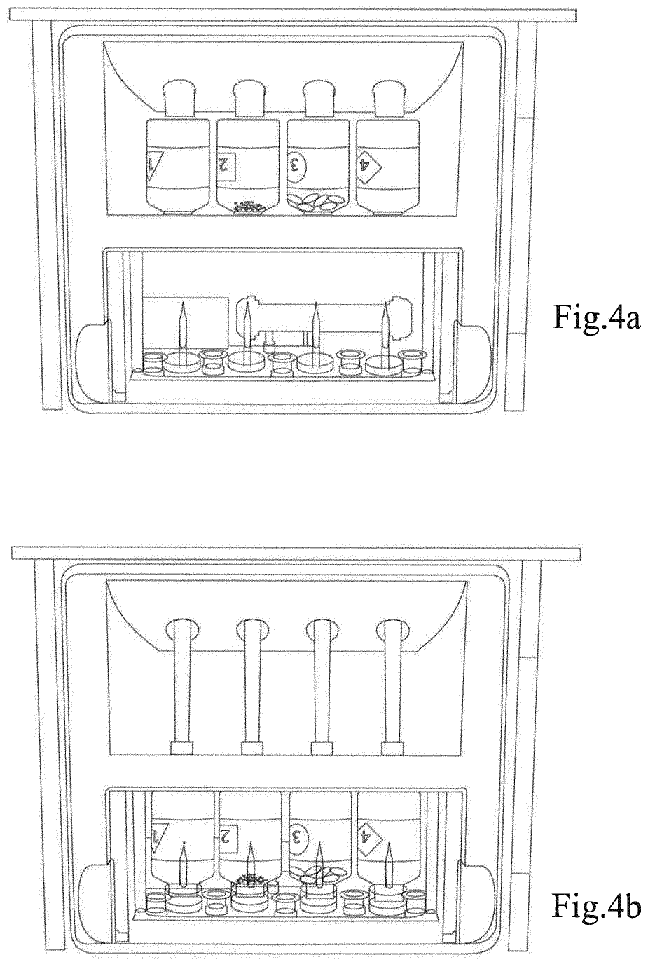

The disposable cartridge 20 which is shown in more details in FIGS. 2 and 3 comprises a rigid tubing circuitry 21 for mixing the components housed in the plurality of vials 31-34 and which is preferably provided within a bottom plate 23 in the manner as a microfluidic cartridge. It also comprises at least two, preferably three, even more preferably four (as shown) vial piercing elements 24, adapted to be inserted within the vial 31-34 to enter in fluid communication with its content and the tubing circuitry 21 is adapted to put at least a first 31 and a second 32 vials in fluid communication so as to mix the contents of the vials together of one of them to the other of them.

As explained above, the mixing is carried out through the use of a pressure gradient generating mechanism 12 which creates either overpressure or depression in at least one of the vials 31-34 or a gas flow in the circuitry 21.

It preferably further comprises a storing vessel 25 for storing an intermediary product obtained through the mixing of at least two vials 31-32. This is particularly useful in case of mixing several reagents at different steps of the compounding in order to obtain a first intermediary product and keep it stored while carrying out a different reaction to obtain a second intermediary product before mixing both intermediary products.

In addition, the disposable cartridge 20 further comprises a microfilter 26 located between the vials 31-32 and a storing vessel 25.

Also, the disposable cartridge 20 comprises at least one buffer tank 27 connected to one vial, here two are represented in FIG. 2 but one can be sufficient. These buffer tanks 27 are designed for storing some foam which is usually generated upon stirring. In fact, when one uses proteins as reagents, foaming is likely to happen, the buffer tanks help in storing this foam outside the vials or at least away from the air inlet.

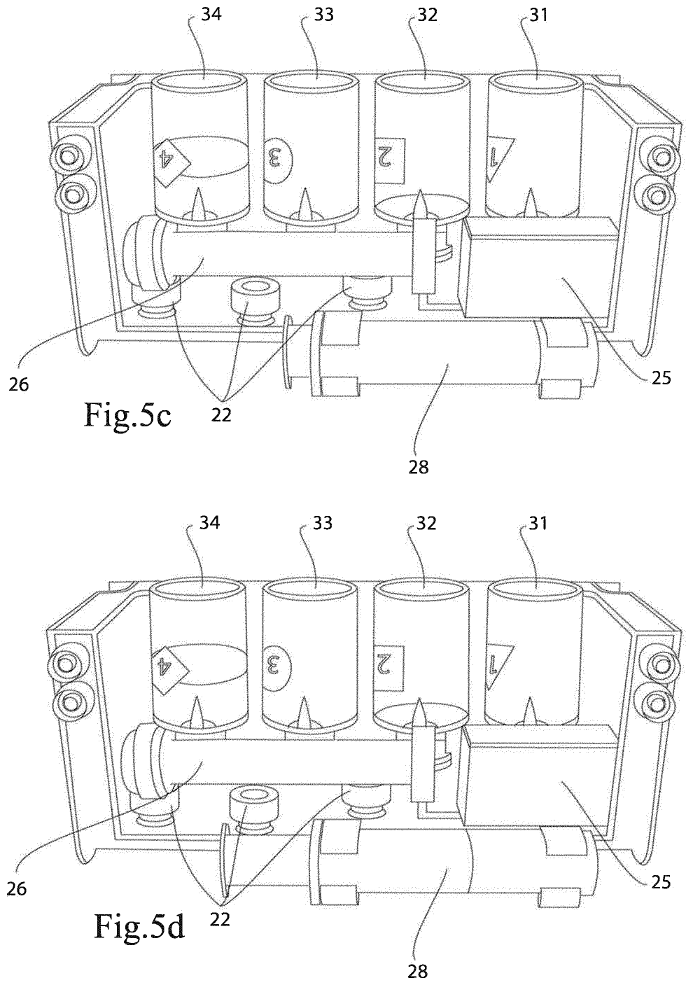

Another aspect of the present invention, illustrated in FIGS. 5a-5d is a component mixing method comprising using the component mixing device according to the above aspect of the invention and carrying out the following steps:

placing a first vial comprising an aqueous solution of glucose, NaSCN and a buffer, and a second vial comprising glucose oxidase and lactoperoxidase,

placing the disposable cartridge 20 into the cavity 14,

controlling said valves 22 and said pressure gradient generating mechanism 12 to
first create a communication path between said first vial 31 and second vial 32 to mix their content in order to carry out a 2-step enzymatic reaction through the use of compressed air to synthetize HOSCN and/or OSCN⁻,
then pass said OSCN⁻ solution through a dialysis microfilter 26 and storing the resulting OSCN⁻ solution in a storing vessel 25.

Optionally, one can use a kit with more than two vials, such as three vials. In this case both the central unit 10 and the disposable cartridge 20 are adapted for three vials instead of two. This means that the software is adapted and that the central unit 10 comprises three vials positioning element and that the disposable cartridge 20 comprises two vials piercing elements.

In this case, in addition to the above, a third vial 33 comprising a lactoferrin component in respective of vial positioning elements 13, and after storing the OSCN⁻, we create a communication path between said third vial 33 and said storing vessel to mix said lactoferrin with said OSCN⁻ solution through the use of compressed air so as to prepare an extemporaneous medicine for treating CF lung infections, and we store said extemporaneous medicine for treating CF lung infections into a final container 28 which can be a syringe in a preferred embodiment.

According to a preferred embodiment, the invention relates to the use of the above described-component mixing device for preparing an extemporaneous drug for treating lung infections preferably in cystic fibrosis patients but also in bronchiectasis (BE) patients and/or COPD patients.

According to the embodiment, a patient uses a plurality of vials that comprises at least three vials which comprise H₂O₂ in the first vial, a peroxidase, thiocyanate and/or iodide, chloride or bromide ion in the second vial. Also, in case of three or four vials kit, lactoferrin is in the third vial. Alternatively, and as shown in all figures, the plurality of vials comprises four vials wherein the first one and the second are identical to the above and the third one comprises solid lactoferrin and the fourth one comprises NaCl in water.

With such embodiment, other antimicrobial compound such as OI⁻, OBr— or mix with OSCN⁻, e.g. OI⁻/OSCN⁻ can be easily produced

The advantage of having four vials instead of three is that with the fourth vial one can vary the lactoferrin concentration in the solution whereas with the three vials, the lactoferrin concentration is fixed and corresponds to the one chosen beforehand.

The component mixing device together with the vials constitutes a component mixing and therefore kit comprises the component mixing device of the first aspect of the invention and a plurality of vials, each vials being adapted to comprise a specific quantity of reagent for compounding a specific drug, in this example, a drug for treating cystic fibrosis lung infections.

The component mixing kit comprises one vial containing two enzymes enzyme, one vial containing a substrate and optionally one third vial containing lactoferrin and being adapted to compound a CF lung infections treating drug.

As explained above, a preferable use of the component mixing kit is for manufacturing an extemporaneous pharmaceutical formulation intended for the treatment of the acute and/or chronic phases of lung infections in cystic fibrosis for example, comprising OSCN— ion and/or HOSCN and lactoferrin wherein the pharmaceutical formulation is in an inhalable form.

While the embodiments have been described in conjunction with a number of embodiments, it is evident that many alternatives, modifications and variations would be or are apparent to those of ordinary skill in the applicable arts. Accordingly, this disclosure is intended to embrace all such alternatives, modifications, equivalents and variations that are within the scope of this disclosure. This is for example particularly the case regarding the exact number of vials, the specific design of the rigid tubing circuitry and the type of component to be mixed.

1-29. (canceled)

30. A disposable cartridge to be used with a separated central unit in a component mixing device, comprising:
a rigid microfluidic circuitry including
at least two circuitry inlets each being adapted to enter in fluid communication with a vial to permit insertion of content of each vial within said circuitry,
at least one circuitry outlet for outputting a mixed product from the disposable cartridge and
a plurality of valves adapted to be controlled by a control unit of said central unit in order to create a specific path within said circuitry between said inlets and said outlet,
wherein said circuitry is adapted to enter in fluid communication with a pressure gradient generating mechanism of said central unit in order to generate a circuitry content driving force within it.

31. The disposable cartridge according to claim 30, wherein said circuitry is adapted to put at least a first vial and a second vial in fluid communication so as to mix reagent content of the first vial with reagent content of the second vial.

32. The disposable cartridge according to claim 30, further comprising at least one storing vessel for storing an intermediary product obtained through the mixing of contents of at least two vials.

33. The disposable cartridge according to claim 30, further comprising a microfilter for filtering at least one intermediary product.

34. The disposable cartridge according to claim 33, wherein the microfilter is located between the inlets and the storing vessel.

35. The disposable cartridge according to claim 33, wherein said microfilter is a dialysis microfilter adapted to switch between tangential and frontal filtration.

36. The disposable cartridge according to claim 30, wherein a mixing process is carried out through the use of a pressure gradient which is generated by a compressed air mechanism located within said central unit which creates either overpressure or depression in at least one of the vials.

37. The disposable cartridge according to claim 30, further comprising at least one buffer tank connected to the vials or the microfilter.

38. The disposable cartridge according to claim 36, wherein the mixing process carried out is selected from the group consisting of a dilution, a reaction, a succession of reactions, an enzymatic cleavage, an elution, and a purification.

39. A component mixing device comprising the disposable cartridge of claim 30 and the central unit of claim 30, wherein the component mixing device is adapted to put specific vials into communication with each other through said circuitry of said disposable cartridge and mixing said contents according to a specified reacting process through a pressure gradient generating mechanism located in said central unit and controlled by said control unit.

40. A component mixing method comprising using the component mixing device according to claim 39 and carrying out the following steps:
placing a first vial comprising an aqueous solution of glucose, NaSCN and a buffer and a second vial comprising glucose oxidase and lactoperoxidase in respective vial positioning elements,
placing the disposable cartridge into a cavity,
controlling said valves and said pressure gradient generating mechanism to
first create a communication path between said first vial and second vial to mix their content in order to carry out a 2-step enzymatic reaction through the use of compressed air to synthetize HOSCN and/or OSCN⁻,
then pass said OSCN⁻ solution through a dialysis microfilter and storing the resulting OSCN⁻ solution in a storing vessel.

41. The component mixing method according to claim 40, wherein the plurality of vials comprises at least three vials where the third vial comprises a lactoferrin component and further comprises the steps of creating a communication path between said third vial and said storing vessel to mix said lactoferrin with said OSCN⁻ solution through the use of compressed air so as to prepare an extemporaneous medicine for treating CF lung infections, and storing said extemporaneous medicine for treating CF and/or BE lung infections into a syringe.

42. The component mixing method according to claim 41, wherein said third vial comprises a NaCl water diluted lactoferrin solution.

43. The component mixing method according to claim 40, wherein the plurality of vials comprises at least four vials wherein the third vial comprises solid lactoferrin and the fourth vial comprises NaCl in water.

44. A component mixing kit comprising the component mixing device according to claim 39 and a plurality of vials, each of the vials being adapted to comprise a specific quantity of reagent for compounding a specific drug.

45. The component mixing kit according to claim 44 comprising one vial containing an enzyme, one vial containing a substrate and one vial containing lactoferrin and being adapted to compound a CF lung infections treating drug.

46. A method comprising using the component mixing kit according to claim 44 to manufacture an extemporaneous pharmaceutical formulation intended for the treatment of mucous microbial infections wherein the pharmaceutical formulation is a fluidic antimicrobial solution.

47. A method comprising using the component mixing kit according to claim 44 to manufacture an extemporaneous pharmaceutical formulation comprising OSCN⁻ ion and/or OF and/or OBr⁻ and/or lactoferrin wherein the pharmaceutical formulation is intended to be used as an antimicrobial agent.

48. A method comprising using the component mixing kit according to claim 44 to treat acute and/or chronic phases of the lung infections wherein the pharmaceutical formulation is in an inhalable form.

49. A method comprising using the component mixing kit according to claim 44 to manufacture an extemporaneous pharmaceutical formulation comprising at least one unstable antimicrobial compound wherein the said antimicrobial compound can be purified and/or combined with another pharmaceutical compound to produce an individualized pharmaceutical product.

50. A method comprising using the component mixing kit according to claim 44 to address unmet medical needs such as treatment of infections due to multidrug resistant microorganism.

51. The component mixing device according to claim 39, wherein the central unit further comprises a plurality of vial positioning elements for receiving vials comprising specific reagents, a cavity for receiving said disposable cartridge, a control unit and a pressure gradient generating mechanism controlled by said control unit, wherein said pressure gradient generating mechanism is adapted to generate overpressure or depression in specific portions of said disposable cartridge to generate a reagent/product driving force.

52. The component mixing device according to claim 39, wherein the central unit further comprises a movement mechanism adapted to move said vials into a cavity so as to enter in fluid communication with each other through said circuitry of the disposable cartridge.

53. The component mixing device according to claim 39, wherein the central unit further comprises in-air filters.

54. The component mixing device according to claim 39, wherein the central unit is adapted to control a desired quantity of each of the contents according to a user's choice.

55. The component mixing device according to claim 39, wherein said central unit further comprises a temperature monitoring system to regulate temperature in a cavity.

56. The component mixing device according to claim 39, wherein the central unit further comprises a screen for displaying successive steps of the mixing process according to claim 36.

57. The component mixing device according to claim 56, wherein said screen is adapted to warn said user upon a wrong placement of a vial.

58. The method according to claim 48, wherein the lung infections are selected from the group consisting of cystic fibrosis and bronchiectasis.
